(19) United States
(12) Patent Application Publication
(10) Pub. No.: US 2004/0225279 A1
(43) Pub. Date: Nov. 11, 2004

(54) Detachable tip microcatheter for use of liquid embolic agents

(76) Inventor: Jean Raymond, Montreal (CA)

Correspondence Address: David S Resnick, Nixon Peabody, 100 Summer Street, Boston, MA, 02110-2131, US

(21) Appl. No.: 10/479,489

(22) PCT Filed: Jun. 3, 2002
(86) PCT No.: PCT/CA02/00815

Related U.S. Application Data
(60) Provisional application No. 60/294,612, filed on Jun. 1, 2001.

(51) Int. Cl. A61M 25/00

(52) U.S. Cl. 604/523; 606/191

(57) ABSTRACT

The present invention relates to microcatheters (8) with a detachable tip for administering liquid embolic agents. The detachable tip microcatheter for use with liquid embolic agents in treating an aneurysm comprises a body adapted to be introduced in a vascular cavity; a detachable tip portion mounted on a distal end of the body; and a detaching mechanism (16) mounted between the tip and the body for detaching the tip from the body, the tip portion being adapted to be positioned in use in the aneurysm to introduce the embolic agent into the aneurysm.

### TECHNICAL FIELD

The present invention relates to microcatheters with a detachable tip for administering liquid embolic agents.

### BACKGROUND OF THE INVENTION

Cyanoacrylates and other liquid embolic agents that polymerize or precipitate inside vessels have long been used in the treatment of vascular diseases. Cyanoacrylates are effective occlusive agents in neurovascular interventions. Cyanoacrylates and other liquid embolic agents may have the potential to improve long-term results of endovascular treatment of aneurysms.

Endovascular treatment of acutely ruptured aneurysms with Guglielmi Detachable Coils (GDCs) is both safe and effective (references 1-3). The main drawback of this approach is the incidence of recurrences, which are more. frequent after embolization than after surgical clipping (references 4-7). Methods to improve the long-term results of embolization include surface modification of coils (references 4, 10, 11), addition of fibers (reference 12), local growth factor delivery (references 13, 14) or embolization with polymers (references 15-18). Since the pioneering work of Zanetti (reference 19), Kerber (reference 20) and Debrun (reference 21), acrylics have never gained wide acceptance in aneurysms, mainly because of the risks of cerebral infarction from uncontrolled escape of the polymers during deposition. An added difficulty is the risk of gluing or cementing the catheter with the embolic material. An aneurysm model prone to recurrences following embolization has been developed (references 8, 9). Endovascular acrylic deposition with microcatheters led to stray emboli in all cases. Acrylic delivery was improved by a single coil positioned at the neck of the aneurysm, but parent vessel embolization still occurred in 25% of animals. Acrylic embolization of bifurcation aneurysms improved angiographic results at 3 months as compared to coil embolization (see Table 1).

TABLE 1
Angiographic scores and neointima thickness in bifurcation
aneurysms treated with coils or acrylic

| | Coils | Acrylic & coils |
|---|---|---|
| | (n = 6) | (n = 6) |
| Initial angiographic score | 0.40 ± 0.89 | 1.00 ± 1.16 |
| Score 3 weeks | 0.75 ± 0.96 | 0.00 ± 0.00 |
| Evolution at 3 weeks | 0.75 ± 0.96 | −1.00 ± 1.16 |
| Score at 3 months | 3.00 ± 0.71 | 0.50 ± 1.00^{a} |
| Evolution at 3 months | 2.60 ± 0.55 | −0.50 ± 1.92^{b} |
| Neointima at 3 months | 29.80 ± 24.36 | 300.00 ± 50.09^{c} |

^{a}= The mean angiographic score of aneurysms treated with acrylic over a coil is smaller than the score in aneurysms treated with coils at 3 months (p = 0.0008)
^{b}= The evolution at 3 months of aneurysms treated with coils is worse than aneurysms treated with acrylic (p = 0.01)
^{c}= The neointima at the neck of aneurysms treated with acrylic behind a coil is significantly thicker than the one found in aneurysms treated with coils (p < 0.05)

With the advent of a number of polymeric glues and glue substitutes for neurologic use for the treatment of diseases like AVM's, aneurysms and other vascular diseases there is a lack of safe and effective methods for the delivery of these substances within the neurovasculature, especially when longer times are required for the effective delivery of certain of these compounds. The problem remains that the polymer tip can become entrapped and glued in place with severe complications for patient. The device of the present invention is designed to overcome these limitations.

It would be highly desirable to be provided with a safe, controllable and effective device for the use of acrylic and other liquid embolic agents in endovascular treatment of vascular pathologies.

### SUMMARY OF THE INVENTION

One aim of the present invention is to provide a safe, controllable and effective device for the use of acrylic and other liquid embolic agents in endovascular treatment of vascular pathologies.

In accordance with the present invention there is provided a detachable tip microcatheter for use with liquid embolic agents in treating an aneurysm, said microcatheter comprising:

a) a body adapted to be introduce in a vascular cavity;

b) a detachable tip portion mounted on a distal end of said body; and

c) a detaching mechanism mounted between said tip and said body for detaching said tip from said body, said tip portion being adapted to be positioned in use in the aneurysm to introduce said embolic agent into said aneurysm.

In a preferred embodiment, the embolic agent is a biocompatible glue or a polymeric agent. The tip portion may be curved to facilitate introduction of said tip in the aneurysm.

In one embodiment of the invention, the detaching mechanism comprises a metal defining a ring between said distal end of the body and said tip portion. The detaching mechanism may further comprise a heating source for heating the metal for releasing the tip portion. Alternatively, the detaching mechanism may comprise a polymer ring between said distal end of the body and said tip portion, said polymer ring being heat-sensitive and breaks upon heating, thereby releasing the tip portion. In another embodiment, the detaching mechanism comprises a polymer ring between said distal end of the body and said tip portion, said polymer ring being electrically cleavable for releasing the tip portion.

The tip portion may as well be a tube for delivering the embolic agent or a balloon, shaped or not. When a balloon is used, the microcatheter is a dual lumen microcatheter if the balloon cannot infuse the embolic agent. Otherwise the balloon is provided with holes, cuts or stripes for infusing the embolic agent.

In one embodiment , the tip portion may be made of polymer. The polymer tip portion may then be detached from the microcatheter using a thermal or chemical source, which when desired or activated, melt the polymer, detaching the tip portion.

### BRIEF DESCRIPTION OF THE DRAWINGS

Having thus generally described the nature of the invention, reference will now be made to the accompanying drawings, showing by way of illustration, a preferred embodiment thereof, and in which:

FIG. 1 illustrates one embodiment of the microcatheter of the present invention;

FIG. 2 illustrates another embodiment of the microcatheter of the present invention;

FIGS. 3A and 3B illustrate a further embodiment of the microcatheter of the present invention;

FIG. 4 illustrates another embodiment of the microcatheter of the present invention;

FIG. 5 illustrates another embodiment of the microcatheter of the present invention;

FIGS. 6A and 6B illustrate another embodiment of the microcatheter of the present invention;

FIGS. 7A to 7C illustrate another embodiment of the microcatheter of the present invention; and

FIGS. 8A to 8D illustrate another embodiment of the microcatheter of the present invention.

It will be noted that throughout the appended drawings, like features are identified by like reference numerals.

### DETAILED DESCRIPTION OF THE PREFERRED EMBODIMENT

In accordance with the present invention, there is provided a new microcatheter with a detachable tip for delivering liquid embolic agents. In one embodiment of the invention, the microcatheter is provided with a tip that can be left in situ, buried within the polymerizing or precipitating mass of the embolic agent. A further preferred characteristic of the microcatheter of the present invention is the possibility of controlling the deposition of the liquid agent as it is polymerizing or precipitating, and at the same time controlling blood flow at the site. The microcatheter of the present invention may in one embodiment be provided with a sealing mechanism for sealing the extremities of the microcatheter and of the detachable tip once the detachable tip has been detached, preventing leaking of a drop of the embolic agent, as the detachable tip is being detached.

The device ensures optimal security for the use of embolic agents in any situation where these agents are potentially useful. In intracranial aneurysms, it permits better filling of the aneurysmal sac, better occlusion of the neck, lesser risks of stray emboli. In arteriovenous malformations or fistulae, it permits better flow control and ease of termination of the embolization, without the risks of “gluing” the entire microcatheter in place, without the need for sudden violent traction at the end of the intervention as is currently the case when acrylics are used.

In one embodiment of the invention, the device of the present invention consists of a microcatheter body built of a coiled or braided microcatheter shaft that has all of the attributes of a microcatheter for flexibility, torque and tracking. The tip consists of a very soft biocompatible polymer (urethane, pebax, PE, nylon, or any number of biodegradable compounds). The detachment zone has a small-embedded nitinol (stainless steel) ring which when activated by either mechanical force or by applied electrical current detaches the tip. The ring is embedded within a soft polymer detachment zone that has only a very thin covering over the ring such that the activated ring can pull through. The activated ring cuts through the polymer tip. Typical activation would occur from the outer surface towards the inner lumen. Alternatively the embedded ring may also be a clamp like structure made of nitinol which when a small electric current is applied opens the ring structure to release an embedded tip. This clamp may be continuous or in several (3-4) discreet points at the tip junction. The detachment could also consist of melting through the tip using a resistive coil or the use of partially conductive polymer with embedded electric wire within the catheter shaft to carry current to the resistive material. The resistive material heats as current passes through melting the polymer tip and detaching the tip. Additionally the detachment of the tip could also seal the device closed via heating of the glue to crosslink it.

Other methods like lasers to melt the tip could also be used or chemical dissolution of a soluble polymeric section could also be used to detach the tip. The delivery of glue with such a device could be done in simple stages where a small amount of glue is injected allowed to harden detach the tip and then bring another device in and repeat the procedure until aneurysmal filling could be completed. This sequential injection could significantly enhance the safety of the delivery of a liquid embolic without requiring a flow arrest device to be used.

The present invention will be more readily understood by referring to the following examples, which are given to illustrate the invention rather than to limit its scope.

### EXAMPLE I

### Aneurysm Glue Retention Device Infusable Core—Aneurysm Coil/Polymeric Filler

With the advent of a number of polymeric glues and glue substitutes for neurologic use for the treatment of aneurysms and other vascular diseases there is a lack of safe and effective methods for the delivery of these substances within an aneurysm especially when longer times are required for the effective delivery of certain of these compounds. This device is designed to overcome these limitations.

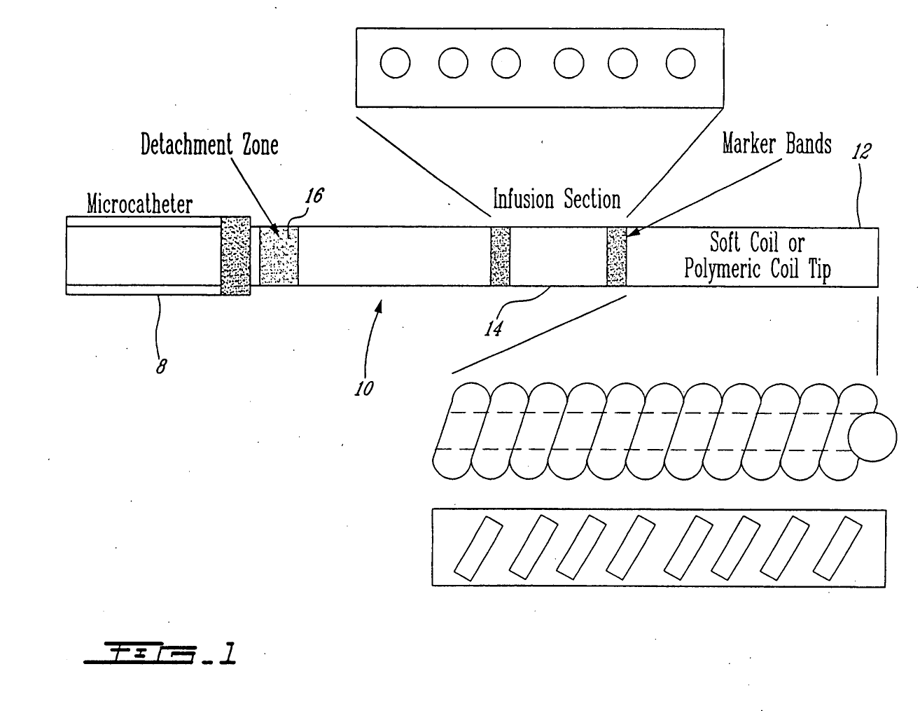

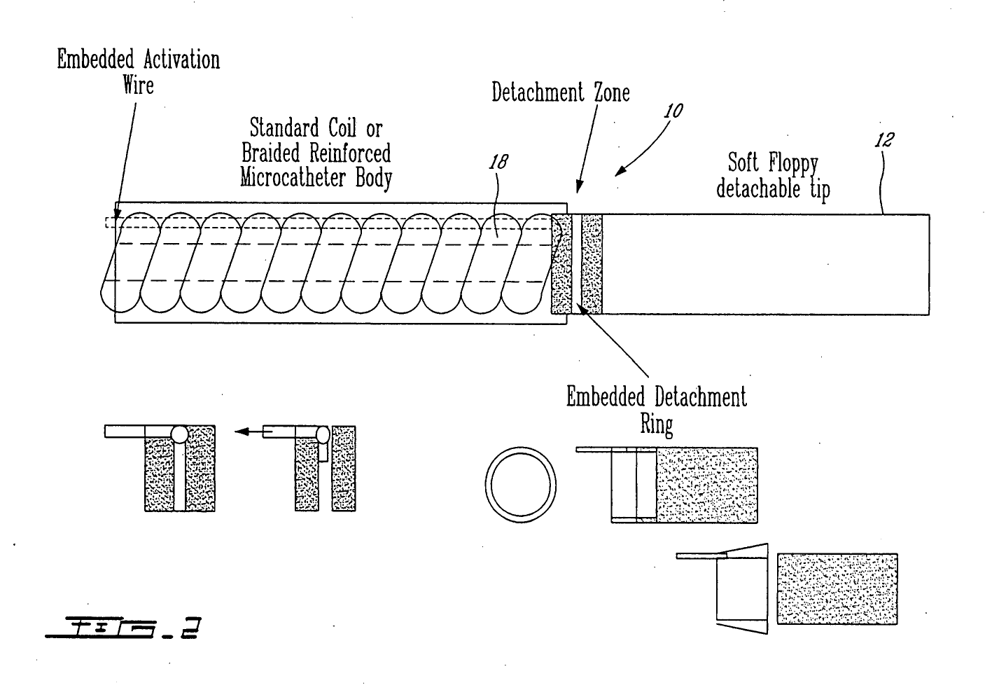

The embodiment illustrated in FIG. 1 would be delivered through a standard microcatheter 8 and via standard neuro-microcatheter access techniques. The device 10 comprises a metallic (Gold, NiTi, Pt, etc.) or polymeric (PE, nylon, urethane, Polyester, etc.) coil tip 12 being long enough to have several loops form within the aneurysm, which would allow for blood flow control into the aneurysm (see FIGS. 1 and 2). This coil tip 12 could also have several polymeric fibers (PGA, PLA, nylon, polypropylene, etc.) embedded within it to help entrap any escaping infusate. The section just behind the coil tip 12 consists of an infusion region 14. This region would be placed near the dome of the aneurysm and glue or the equivalent would then be infused out of this infusion region 14 and allowed to slowly polymerize within the coil loops or basket. The entire device 10 could then be detached through a variety of means (laser, heat, electrical, chemical or mechanical as described herein). The device body through which the infusion would take place could be a polyimide tube, metallic hypo-tube, or the equivalent that would have a higher melt temperature than the polymeric infusion zone and the distal portion of the device leading up to the connection point or detachment zone 16 so that detachment by heating would be easy. Additionally if a small heater element is passed through the device for the detachment of the device and heated for detachment this would also seal the device closed via heating of the glue to crosslink it.

### EXAMPLE II

### Aneurysm Glue Retention Device Expanding Cone Tip Catheter—Chemical

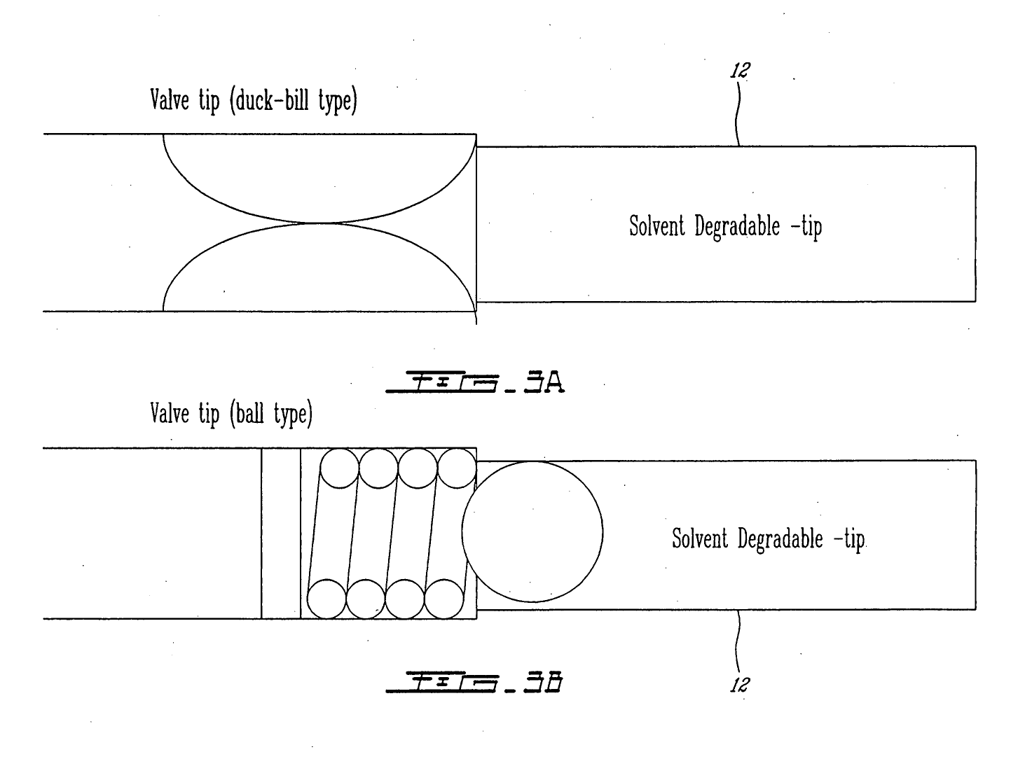

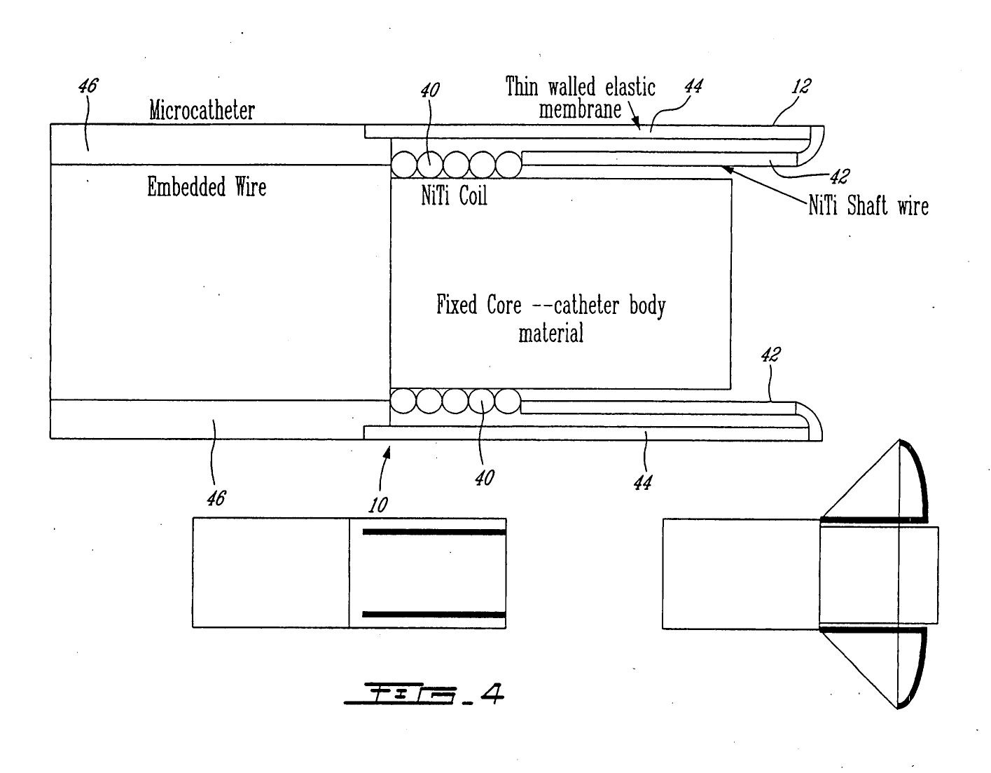

Chemical dissolution of a soluble polymeric section could also be used to detach the tip 12 as illustrated in FIGS. 3A and 3B. Injection of glue with base solvent, solvent alone, or solvent injected into separate lumen running only to the detachment zone could be used to introduce a solvent which could easily dissolve the soft polymer tip at a discreet location through the use of a special zone of dissolvable polymer. The use of valving on the tip could ensure dissolution at a specific site or seal the solvent within this zone to allow better dissolving of the polymer section.

### EXAMPLE III

### Aneurysm Glue Retention Device Expanding Cone Tip Catheter—Electric

In FIG. 4, the device 10 comprises a microcatheter 8 body and is built of a coiled or braided microcatheter shaft that has all of the attributes of a microcatheter for flexibility, torque and tracking. The tip 12 however is specially designed to flare out when placed at the neck of the aneurysm and exclude it from flow while coils and/or glue can be placed within the aneurysm itself. One preferred design consists of nitinol coil 40 with discreet nitinol wire shafts 42 covered by a thin elastic membrane 44 (urethane, silicones, elastomerics, etc.). The nitinol has a small current applied to it from wires 46 embedded within the catheter body which causes the coils to elongate along the infusion shaft which pushes the constrained coil and the attached shaft wire outward. This creates a cone-like tip with a central fixed infusion lumen open for coil delivery or glue delivery or a combination of both. Alternatively the coil could contract when current passes through it causing the constrained shaft wire to bow outward to create the cone.

### EXAMPLE IV

### Aneurysm Glue Retention Device Expanding Cone Tip Catheter—Mechanical

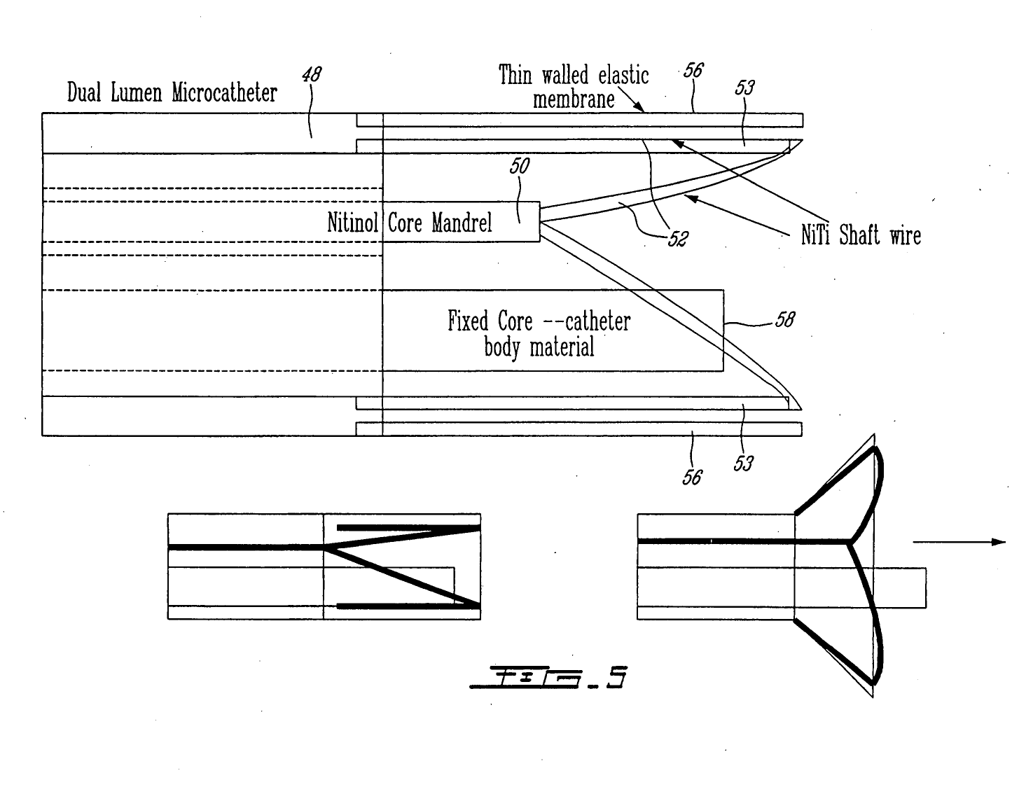

One alternative embodiment of the above design is a mechanically expanded cone tip 12 (see FIG. 5). The invention comprises a dual lumen based catheter 48 with one lumen given to a core wire and/or mandrel 50 (nitinol). The core mandrel 50 has wires 52 welded to the tip and connected to a flexible shaft wire 53 embedded in the thin elastic membrane material 56 (urethane, silicones, elastomerics, etc.). Mechanical movement of the mandrel 54 will push (or alternatively pull) the shaft wires 53 causing them to bow outwardly, thus creating a cone tip. The second lumen is a fixed infusion lumen 58 open for coil delivery or glue delivery or a combination of both.

### EXAMPLE V

### Aneurysm Glue Retention Device Side-hole Infusion Balloon

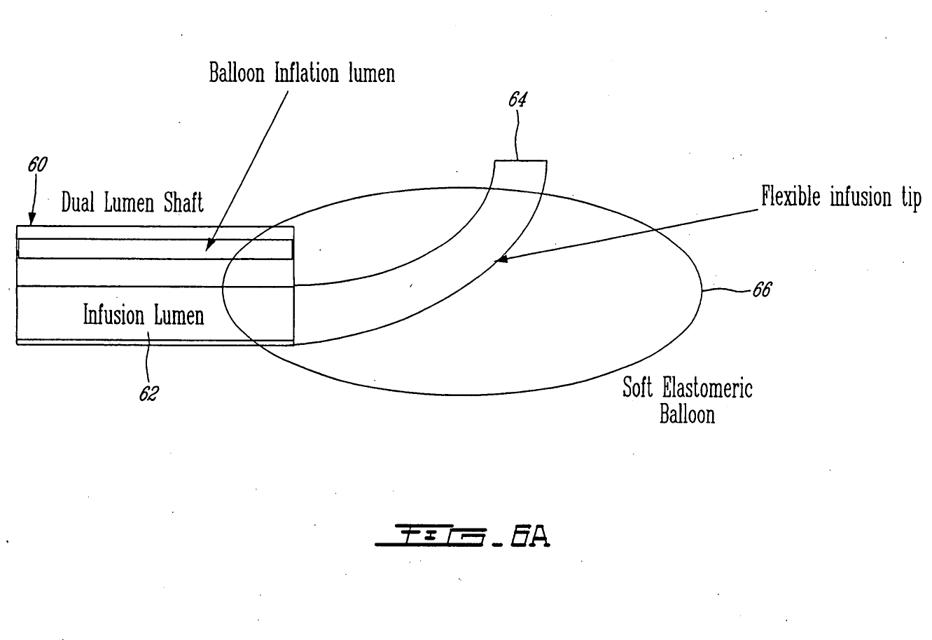

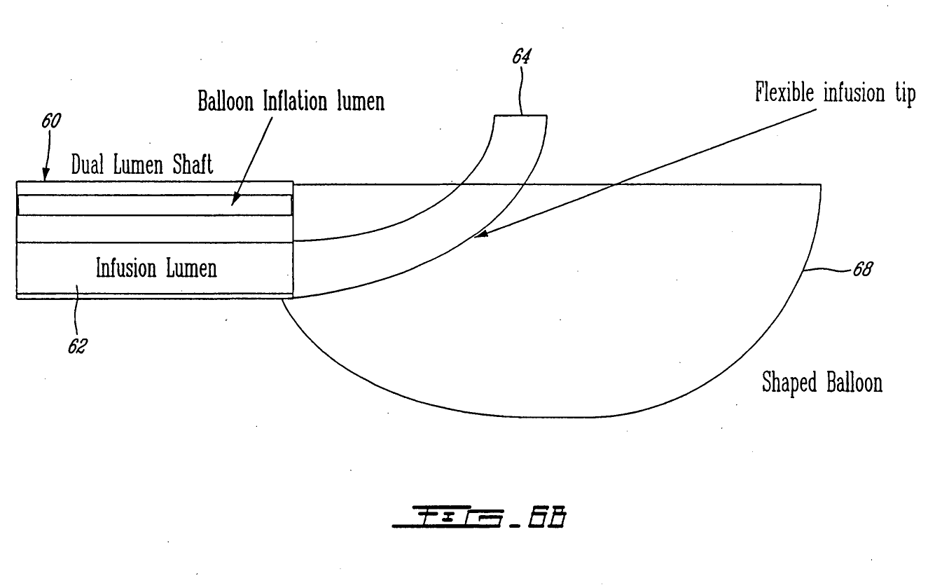

In this embodiment of the present invention, the microcatheter comprises a dual lumen balloon catheter 60 (see FIGS. 6A and 6B). An infusion lumen 62 is tipped with a soft elastic tip 64, which is bonded to the side of the balloon with appropriate radiographic markings. The balloon can either be an ultra-soft elastomeric balloon 66 (urethane, silicones, etc) (FIG. 6A) or a shaped balloon 68 (PE, polyester, etc.) (FIG. 6B) with the infusion lumen 62 attached to the flattened side of the shaped balloon.

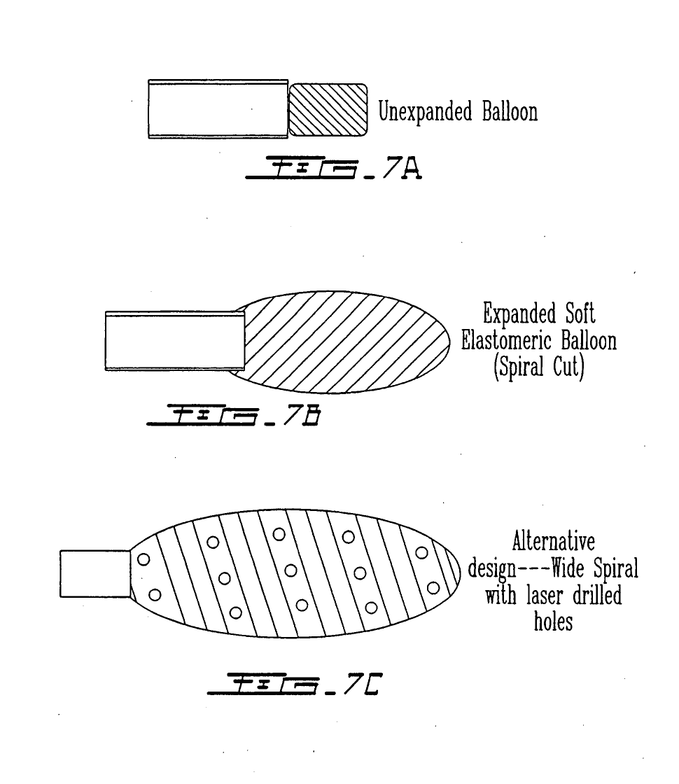

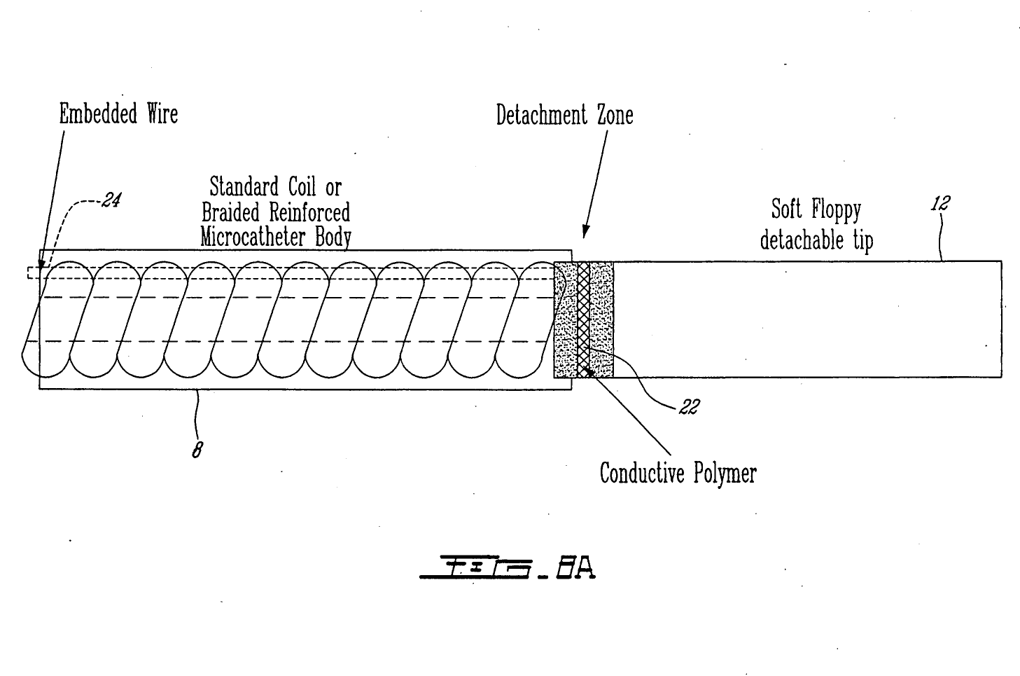

FIGS. 7A to 7C illustrate various balloons that can be used with the present invention. In FIG. 7A, the balloon is illustrated as unexpanded, whereas in FIG. 7B, the balloon is a soft elastomeric balloon with spiral cut that has been inflated. FIG. 7C illustrates an alternative of the balloon illustrated in FIG. 7B, whereas the balloon has a wide spiral design with laser drilled holes.

### EXAMPLE VI

### Electrical Detachment of a Tip of a Catheter

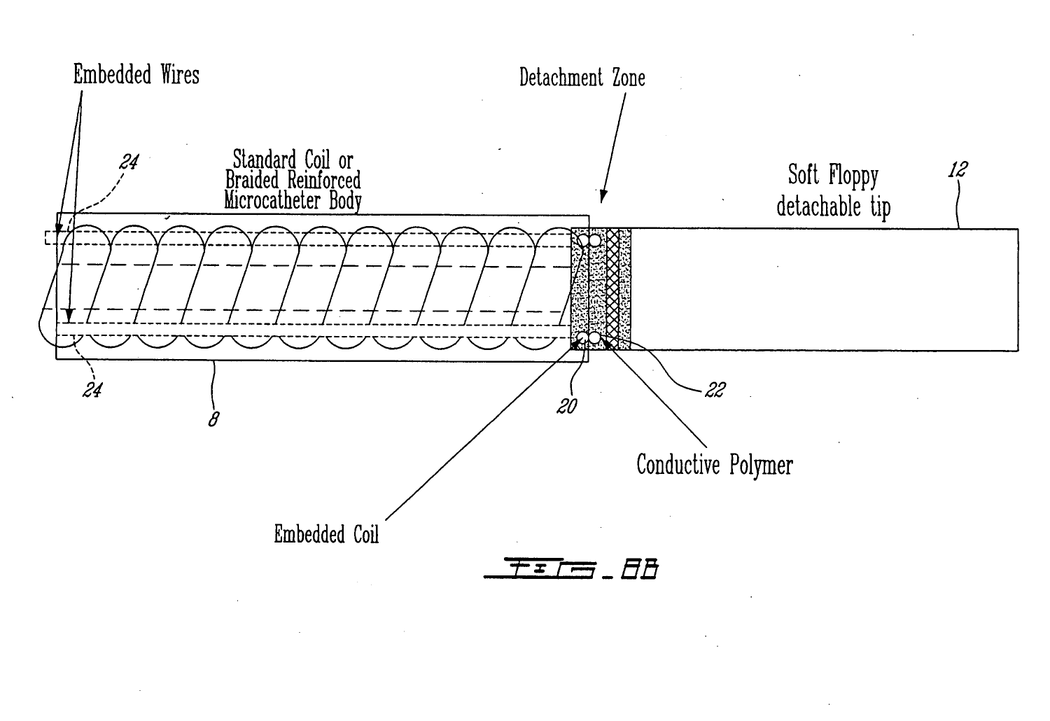

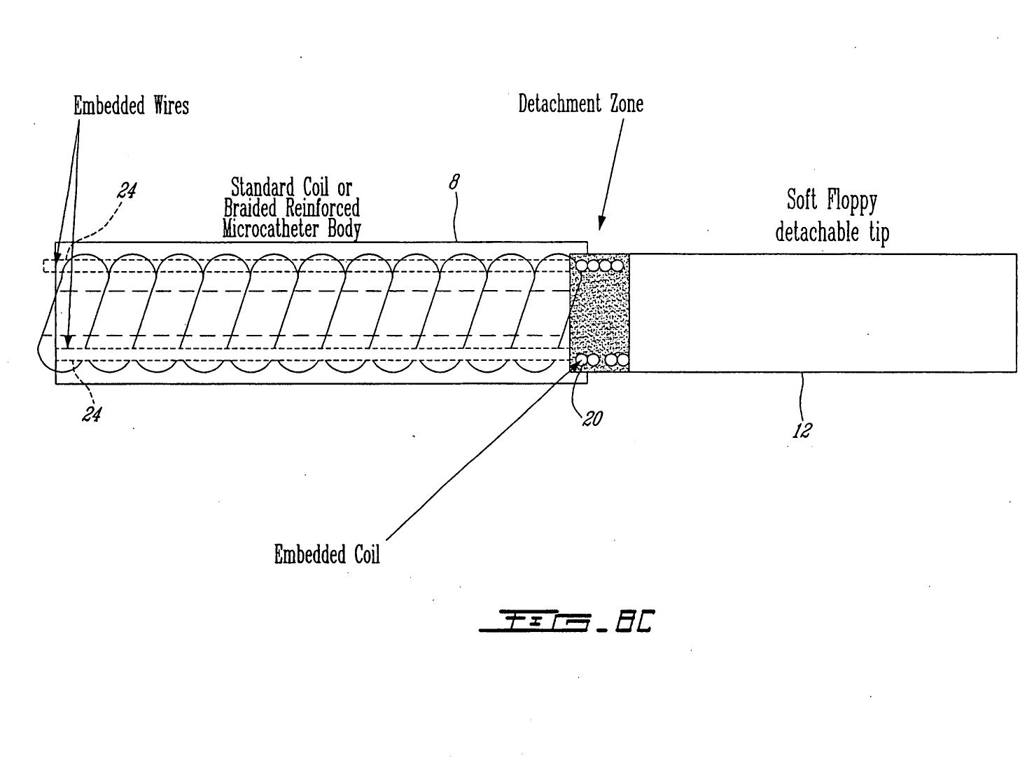

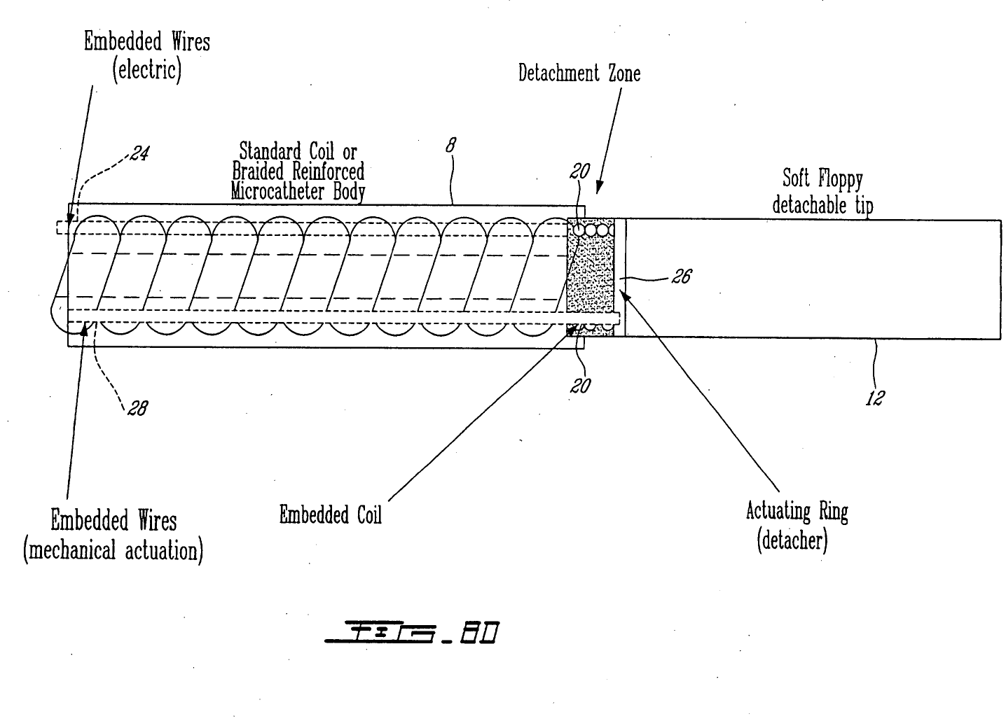

As illustrated in FIGS. 8A to 8D, the detachment could also consist of melting through the tip 12 using a resistive coil 20 or the use of partially conductive polymer or any combination of either with embedded electric wire 24 within the catheter shaft to carry current to the resistive material. The resistive material heats as current passes through melting the polymer tip and detaching the tip 12. Additionally the detachment of the tip 12 could also seal the device closed via heating of the glue to crosslink it. This could also be paired with a mechanical detachment system 26 wherein the heater partially melts or softens the polymer to enable ease of mechanical detachment. The mechanical system could consist of an embedded ring 26 which when actuated, decreases in diameter. The ring 26 can be attached with embedded wires 30. When a current is applied to the ring 26, the ring contracts reducing its diameter. Other methods like lasers to melt the tip could also be used.

While the invention has been described with particular reference to the illustrated embodiment, it will be understood that numerous modifications thereto will appear to those skilled in the art. Accordingly, the above description and accompanying drawings should be taken as illustrative of the invention and not in a limiting sense.

### References

1. Guglielmi detachable coil embolization of acute intracranial aneurysm: perioperative anatomical and clinical outcome in 403 patients. Viñuela F, Duckwiler G, Mawad M, J. Neurosurg 1997 Mar 86:475-82 ;

2. Byrne et al., Neuroradiology, 36 :165-166, 1994;

3. Safety and efficacy of endovascular treatment of acutely ruptured aneurysms. Raymond J, Roy D., Neurosurgery 1997 Dec 41:6 1235-45;

4. Ion implantation and protein coating of detachable coils for endovascular treatment of cerebral aneurysms: concepts and preliminary results in swine models. Murayama Y, Vinuela F, Suzuki Y, Do H M, Massoud T F, Guglielmi G, Ji C, Iwaki M, Kusakabe M, Kamio M, Abe T., Neurosurgery 1997 Jun 40:6 1233-43; discussion 1243-4;

5. Endovascular treatment of acutely ruptured and unruptured aneurysms of the basilar bifurcation. Raymond J, Roy D, Bojanowski M, Moumdjian R, L_Esperance G., J Neurosurg 1997 Feb 86:2 211-9;

6. Natural history of postoperative aneurysm rests. Feuerberg I, Lindquist C, Lindqvist M, Steiner L., J Neurosurg 1987 Jan 66:1 304;

7. Regrowth of aneurysm sacs from residual neck following aneurysm clipping. Lin T, Fox A J, Drake C G., J Neurosurg 1989 Apr 70:4 556-60;

8. Healing mechanisms in experimental aneurysms. I. Vascular smooth muscle cells and neointima formation. Raymond J, Venne D, Alias S, Roy D, Oliva V L, Denbow N, Salazkin I, Leclerc G., J Neuroradiol 1999 Mar 26:1 7-20;

9. Fibrinogen and vascular smooth muscle cell grafts promote healing of experimental aneurysms treated by embolization. Raymond J, Desfaits A C, Roy D., Stroke 1999 Aug 30:8 1657-64;

10. Radiologic and histopathologic evaluation of canine artery occlusion after collagen-coated platinum microcoil delivery. Tamatani S, Ozawa T, Minakawa T, Takeuchi S, Koike T, Tanaka R., AJNR Am J Neuroradiol 1999 Apr 20:4 541-5;

11. Treatment of experimental aneurysms using collagen-coated microcoils. Dawson R C, Krisht A F, Barrow D L, Joseph G J, Shengelaia G G, Bonner G., Neurosurgery 1995 Jan 36:1 133-9; discussion 139-40;

12. Endovascular packing of carotid bifurcation aneurysm with polyester fiber-coated platinum coils in a rabbit model. Kwan E S, Heilman C B, Roth P A., AJNR Am J Neuroradiol 1993 Mar-Apr 14:2 323-33;

13. Healing of experimental aneurysms. II: Platelet extracts can increase the thickness of the neointima at the neck of treated aneurysms. Venne D, Raymond J, Allas S, Roy D, Leclerc G, Boushira M, Brazeau P., J Neuroradiol 1999 Jun 26:2 92-100;

14. Growth factors stimulate neointimal cells in vitro and increase the thickness of the neointima formed at the neck of porcine aneurysms treated by embolization. Desfaits A C, Raymond J, Muizelaar J P., Stroke 2000 Feb 31:2 498-507;

15. Partial thrombosis of canine carotid bifurcation aneurysms with cellulose acetate polymer. Tokunaga K, Kinugasa K, Mandai S, Handa A, Hirotsune N, Ohmoto T., Neurosurgery 1998 May 42:5 1135-42; discussion 11424;

16. Experimental aneurysms in the rabbit: occlusion by intrasaccular injection of fibrin sealant. Moringlane J R, Grote R, Vonnahme F J, Mestres P, Harbauer G, Ostertag C B., Surg Neurol 1987 Nov 28:5 361-6;

17. Randomized comparison of Guglielmi detachable coils and cellulose acetate polymer for treatment of aneurysms in dogs. Macdonald R L, Mojtahedi S, Johns L, Kowalczuk A., Stroke 1998 Feb 29:2 478-86;

18. Nonadhesive liquid embolic agent for cerebral arteriovenous malformations: preliminary histopathological studies in swine rete mirabile. Murayama Y, Vinuela F, Ulhoa A, Akiba Y, Duckwiler G R, Gobin Y P, Vinters H V, Greff R J., Neurosurgery 1998 Nov 43:5 1164-75;

19. Experimental evaluation of a tissue adhesive as an agent for the treatment of aneurysms and arteriovenous anomalies. Zanetti P H, Sherman F E., J Neurosurg 1972 Jan 36:1 72-9;

20. Experimental carotid aneurysms: Part 2. Endovascular treatment with cyanoacrylate. Kerber C W, Cromwell L D, Zanetti P H., Neurosurgery 1985 Jan 16:1 13-7;

21. Obliteration of experimental aneurysms in dogs with isobutyl-cyanoacrylate. Debrun G M, Varsos V, Liszczak T M, Davis K R, Heros R S, Zervas N T., J Neurosurg 1984 Jul 61:1 37-43;

22. Long-term angiographic and histopathologic findings in experimental aneurysms of the carotid bifurcation embolized with platinum and tungsten coils. Reul J, Weis J, Spetzger U, Konert T, Fricke C, Thron A., AJNR Am J Neuroradiol 1997 Jan 18:1 35-42;

23. Treatment of carotid artery aneurysms with platinum coils: an experimental study in dogs. Graves V B, Partington C R, Rufenacht D A, Rappe A H, Strother C M., AJNR Am J Neuroradiol 1990 Mar-Apr 11:2 249-52;

24. Canine model of terminal arterial aneurysm. Graves V B, Ahuja A, Strother C M, Rappe A H., AJNR Am J Neuroradiol 1993 Jul-Aug 14:4 801-3;

25. Histological findings in giant aneurysms treated with Guglielmi detachable coils. Report of two cases with autopsy correlation.

Molyneux A J, Ellison D W, Morris J, Byrne J V., J Neurosurg 1995 Jul 83:1 129-32;

26. Long-term histopathologic findings in two cerebral aneurysms embolized with Guglielmi detachable coils. Castro E, Fortea F, Villoria F, Lacruz C, Ferreras B, Carrillo R., AJNR Am J Neuroradiol 1999 Apr 20:4 549-52;

27. Gross and microscopic histopathological findings in aneurysms of the human brain treated with Guglielmi detachable coils. Bavinzski G, Talazoglu V, Killer M, Richling B, Gruber A, Gross CE, Plenk H., J Neurosurg 1999 Aug 91:2 284-93;

28. The nature of thrombosis induced by platinum and tungsten coils in saccular aneurysms. Byrne J V, Hope J K, Hubbard N, Morris J H., AJNR Am J Neuroradiol 1997 Jan 18:1 29-33;

29. Morphologic characteristics of lesion formation and time course of smooth muscle cell proliferation in a porcine proliferative restenosis model. Carter A J, Laird J R, Farb A, Kufs W, Wortham D C, Virmani R., J Am Coll Cardiol 1994 Nov 1 24:5 1398-405;

30. The restenosis paradigm revisited: an alternative proposal for cellular mechanisms. Schwartz R S, Holmes D R, Topol E J., J Am Coll Cardiol 1992 Nov 1 20:5 1284-93;

31. The intima. Soil for atherosclerosis and restenosis. Schwartz S M, deBlois D, O_Brien E R., Circ Res 1995 Sep 77:3 445-65;

32. Balloon-assisted Guglielmi detachable coiling of wide-necked aneurysms: Part I—experimental evaluation. Akiba Y, Murayama Y, Vinuela F, Lefkowitz M A, Duckwiler G R, Gobin Y P., Neurosurgery 1999 Sep 45:3 519-27;

33. [Reconstruction technic in the treatment of wide-neck intracranial aneurysms. Long-term angiographic and clinical results. Apropos of 56 cases] Moret J, Cognard C, Weill A, Castaings L, Rey A., J Neuroradiol 1997 Jun 24:1 30-44;

34. Endovascular treatment of experimental aneurysms with liquid polymers: the protective potential of stents. Szikora I, Guterman LR, Standard S C, Wakhloo A K, Hopkins L N., Neurosurgery 1996 Feb 38:2 339-47;

35. [An experimental study of the treatment of aneurysms using an intravascular prosthesis] Turjman F, Acevedo G, Massoud T F, Moll T, Sindou M, Guglielmi G, Vinuela F, Froment J C., J Neuroradiol 1997 Oct 24:3 205-11;

36. Mandai S., et al., Interventional Neuroradiol., 4:173-177,1998;

37. Effects of size and shape (aspect ratio) on the hemodynamics of saccular aneurysms: a possible index for surgical treatment of intracranial aneurysms. Ujiie H. Tachibana H, Hiramatsu O, Hazel A L, Matsumoto T, Ogasawara Y, Nakajima H, Hori T, Takakura K, Kajiya F., Neurosurgery 1999 Jul 45:1 119-29; discussion 129-30;

38. Endovascular occlusion of intracranial aneurysms with electrically detachable coils: correlation of aneurysm neck size and treatment results. Fernandez Zubillaga A, Guglielmi G, Vinuela F, Duckwiler G R., AJNR Am J Neuroradiol 1994 May 15:5 815-20.

What is claimed is:

1. A detachable tip microcatheter for use with liquid embolic agents in treating an aneurysm, said microcatheter comprising:
a) a body adapted to be introduce in a vascular cavity;
b) a detachable tip portion mounted on a distal end of said body; and
c) a detaching mechanism mounted between said tip and said body for detaching said tip from said body, said tip portion being adapted to be positioned in use in the aneurysm to introduce said embolic agent into said aneurysm.

2. The microcatheter of claim 1, wherein the embolic agent is a biocompatible glue or a polymeric agent.

3. The microcatheter of claim 1, wherein said tip portion is curved to facilitate introduction of said tip in the aneurysm.

4. The microcatheter of claim 1, wherein said detaching mechanism comprises a metal defining a ring between said distal end of the body and said tip portion and a heating source for heating the metal for releasing the tip portion.

5. The microcatheter of claim 4, wherein the ring is a nitinol ring or a stainless steel ring.

6. The microcatheter of claim 1, wherein said detaching mechanism comprises a polymer ring between said distal end of the body and said tip portion, said polymer ring being heat-sensitive and breaks upon heating, thereby releasing the tip portion.

7. The microcatheter of claim 1, wherein said detaching mechanism comprises a polymer ring between said distal end of the body and said tip portion, said polymer ring being electrically cleavable for releasing the tip portion.

8. The microcatheter of claim 1, wherein the tip portion comprises a balloon.

9. The microcatheter of claim 8, wherein the balloon has holes, cuts or stripes for infusing the embolic agent.

10. The microcatheter of claim 8, wherein the balloon is a shaped balloon.

11. The microcatheter of claim 1, wherein the tip portion is made of polymer.

12. The microcatheter of claim 1, wherein the detaching mechanism is a thermal or chemical source, which when desired or activated, melt the polymer, detaching the tip portion.
